(19) United States
(12) Patent Application Publication
(10) Pub. No.: US 2008/0260880 A1
(43) Pub. Date: Oct. 23, 2008

(54) Oral Compositions for the Prevention of Uv Damages

(76) Inventors: Tomohiro Yokota, Kanagawa (JP); Miki Taniguchi, Kanagawa (JP); Tamami Satou, Kanagawa (JP); Takeshi Ikemoto, Kanagawa (JP)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/884,065

(22) PCT Filed: Feb. 7, 2006
(86) PCT No.: PCT/EP06/01048

§ 371 Date: Mar. 28, 2008

(30) Foreign Application Priority Data
Feb. 10, 2005 (JP) 2005-035234

(51) Int. Cl. A61K 36/185

(52) U.S. Cl. 424/777

(57) ABSTRACT

The invention relates to oral compositions for the prevention of UV damages, in particular to oral compositions based on an olive extract obtained by extracting vegetation water from olive pressing with an organic solvent or by extracting olive cake with water and/or an organic solvent.

### FIELD OF THE INVENTION

The present invention relates to oral compositions for the prevention of UV damages, in particular to oral compositions based on an olive extract obtained by extracting vegetation water from olive pressing with an organic solvent or by extracting olive cake (i.e. the solid phase remained after pressing olives, also called pomace or sansa) with water and/or an organic solvent.

### STATE OF THE ART

When the skin is exposed to UV rays, various damages such as erythema and edema and photo-aging phenomena such as skin thickening, loss of elasticity, formation of wrinkles and skin darkening are caused. Repeated exposure to intense UV rays is known to increase the risk of skin cancer. To prevent UV damages, solar creams are usually employed; however, the application of solar creams might be troublesome, as repeated applications are necessary to provide adequate protection, especially after swimming or excessive perspiration. Therefore, there is still the need for a convenient and effective preparation for the prevention UV damages.

Various studies have been carried out in order to find out orally administrable physiological ingredients effective in protecting the skin from UV rays. For instance, there is evidence that oral administration of carotenoids or Vitamin E can suppress skin inflammation (erythema) caused by UV-rays (Proceedings of Society of Experimental Biology & Medicine, Vol. 223, 170-174, 2000; American Journal of Clinical Nutrition, Vol. 71, 795-798, 2000)

It has also been found that olive extracts (Olea europaea L.), have anti-oxidizing properties, inhibit excessive melanin production and tumor-cell proliferation and also scavenge tumour-cells (JP-A No. 09-78061; WO01/45514, JP-A No. 2002-186453).

Patent applications JP-A No. 2000-319161, JP-A No. 2001-206822 and JP-A No. 2001-252054 disclose a skin cosmetic, a hair tonic and an oral composition containing vegetation water obtained from olive fruits. It has also been found that, when an extract of olive vegetation water or olive cake is orally administered to rats, the anti-oxidation activity of blood plasma is activated and DNA oxidative injury markers induced by sidestream smoke are diminished (Free Rad. Res., Vol. 34, 301-305, 2001; Circulation, Vol. 102, 2169-2171, 2000).

However, the effect of olive extracts from olive pressing residues on the human skin exposed to UV rays has not yet been evaluated.

### DESCRIPTION OF THE INVENTION

It has now been found that extracts obtained from vegetation water and olive cake from olive pressing (hereinafter referred to as “olive fruits extracts”) can prevent UV damages when administered orally, in particular they can prevent erythema, edema, skin thickening, elasticity loss, formation wrinkles and skin darkening when administered through the oral route.

Accordingly, the present invention relates to the use of olive fruits extracts for the preparation of oral compositions for the prevention of UV damages. For the purposes of the present invention, the expression “olive fruit extracts” refers to extracts obtained by extracting vegetation water from olive pressing with an organic solvent or olive cake with water and/or an organic solvent.

The content of “olive fruit extract” in the oral compositions ranges from 0.01 to 70% by weight (dry weight); to inhibit skin damages caused by UV radiation, the composition will be administered so as to provide a dose of “olive fruit extract” in the range of 0.05 to 1.0 g (dry weight) daily.

The olive fruit extracts of the invention can be obtained from olive pressing residues of any kind of olive fruits, irrespective of their provenience or intended use (table olives or oil olives). However, the Coratina variety is particularly preferred. Olive pressing residues are usually discarded, therefore they are relatively cheap.

The extracts may derive from residues of the whole fruits (peel, pulp and seeds) or from the pulp only, after removal of the skin and pulp.

Vegetation water is the aqueous solution obtained as a by-product from olive pressing in the preparation of olive oil. Vegetation water can be used as such; however, lipid, fibrous materials and seed shells normally contained therein are preferably removed by filtration and/or centrifugation. Furthermore, in order to inhibit bacterial contamination and foul smell, hydrophilic alcohols and polyhydric alcohols such as ethyl alcohol, isopropyl alcohol, 1,3-butylene glycol and propylene glycol are added to vegetation water, preferably in the range of 5 to 80% by weight, more preferably in the range of 10 to 40% by weight of the total amount, followed by filtration and centrifugation. Moreover, vegetation water, either as such or after treatment by filtration centrifugation or addition of alcohols, can be concentrated or dried.

“Olive cake” refers to the solid phase obtained by olive pressing.

The extract of the invention can be obtained by extracting vegetation water with an organic solvent or by extracting olive cake with water and/or an organic solvent. Preferred solvents are alcohols, hydrophilic alcohols and polyhydric alcohols such as ethyl alcohol, isopropyl alcohol, 1,3-butylene glycol and propylene glycol. Furthermore, solvent mixtures of water and the organic solvents can also be used. The resulting extracts may be used as such, or concentrated and dried after isolation and purification.

An extract obtained according to the above mentioned extraction method from the solid phase can be used analogously to an extract obtained by extraction of an aqueous phase, or an extract obtained by extraction of an aqueous phase and solid phase.

The extracts of the invention can be added with other active substances, like vitamins such as vitamin C, vitamin E, vitamin B2, vitamin B6 and nicotinic acid amide; minerals such as magnesium, zinc and chromium; Lagerstroemia speciosa, Gymnema sylvestre, Aloaceae, Siraitia Grosvenorii, Zizania latifolia, Morus alba leaf, Eriobotrya japonica leaf, Nelumbo nucifera, Salacia spp., Rhodiola sacrs, indigestible dextrin, Echevaria glauca, green tea polyphenols, theanine, histidine, Panax ginseng, seaweed, hop, Ipomoea batata or beer enzyme. Furthermore, emulsifiers, dispersing agents, suspending agents, spreading agents, penetrating agents, wetting agents and a stabilizing agents may be added. The oral compositions of the invention may be in the solid or liquid form such as tablets, granules, capsules, beverages, jellies, chewing gums, candies and tablet candies.

The amount of “olive fruit extract” varies according to the final administration form; however, in general, in terms of dry weight, the olive extract is preferably contained in the range of 0.01 to 70% by weight of the total weight composition and preferably in the range of 0.01 to 50% by weight. Extract amounts lower than 0.01% do not always provide a sufficient UV damage preventive effect.

The oral composition according to the invention should be administered so as to provide a dose of “olive fruit extract” (dry weight) ranging from 0.05 to 1.0 g a day, preferably from 0.08 to 0.5 g a day. At such doses, the UV damage preventive effect, is sufficient and the compositions can be taken without difficulty; the treatment usually lasts one week or more, according to the subject's needs.

### BRIEF DESCRIPTION OF THE DRAWING

FIG. 1 is a diagram showing the MED variation before and after the continuous ingestion of tablets according to example 1.

The invention will be now illustrated in greater detail by means of some examples.

### EXAMPLES

### Preparation Example 1

### Preparation of an Aqueous Solution and a Concentrate thereof from Olive Fruits Pressing

To 8 L of an aqueous solution obtained in an olive oil manufacturing process from Coratina olive fruits, 2 L of pure ethanol was added. The resulting aqueous-ethanol solution was centrifuged at 4° C. and at 10,000 rpm for 15 min to give substantially 1.5 kg of a solid phase and substantially 8.5 L of an aqueous phase. The aqueous phase was filtered according to a standard process on Celite, affording substantially 8.5 L of a light brown aqueous solution (“aqueous solution of Preparation Example 1”). 5 L of this solution was concentrated according to a standard process to give substantially 220 g of concentrate (“concentrate of Preparation Example 1”).

### Preparation Example 2

### Preparation of a Dry Solid from the Aqueous Solution Obtained by Olive Pressing

74.8 g of the “concentrate of Preparation Example 1” was freeze-dried to obtain 34.84 g of dry solid matter (“dry solid matter of Preparation Example 2”).

### Preparation Example 3

### Preparation of an Extract from the Aqueous and Solid Phase from Olive Pressing

Two kilograms of Coratina variety olive fruits was pressed and extracted twice with aqueous ethanol. The resulting extract was concentrated according to a conventional procedure and 100 g of dry solid matter was obtained (“dry solid matter of Preparation Example 3”).

### Example 1

### Tablets Containing the Dry Solid Matter of Preparation Example 3

Tablets containing the dry solid matter of Preparation Example 3 and the ingredients reported below were prepared according to a standard method.

| | Amount |
|---|---|
| Ingredients | (weight %) |
| (1) “Dry solid matter of preparation example 3” | 7.0 |
| (2) Dextrin | 33.0 |
| (3) Reduced maltose starch syrup powder*¹ | 30.0 |
| (4) Crystalline cellulose | 23.0 |
| (5) Agar powder | 4.0 |
| (6) Aroma | 1.0 |
| (7) Sucrose fatty acid ester | 2.0 |

*¹Trade Name: Malbit, prepared by NIKKEN Fine Chemical Co.Ltd.

### Example 2

### Test for Prevention of Erythema Induced by UV Irradiation Test Procedure

1. 13 healthy males were irradiated on their back and the minimum erythema dose (MED) for each subject was measured. 10 areas of 7.5 mm×7.5 mm were chosen as the UV-irradiating portion. UV rays were irradiated with a (trade name: M-DMR-100, prepared by Clinical Supply Corp.) as a UV-irradiating device, with a UVB: FL20S/E (prepared by TOREX CORP.) and a UVA: S/BL (prepared by TOREX Corp.) arranged in parallel. The intensity of the UV rays was measured with a UV-meter (trade name: UVR-305/360-D (II), prepared by TOREX Corp.) and was found to be 0.45 mW/cm² for the UVB. UV rays were irradiated on the UV-irradiating portions with a varying irradiating period and 24 hr after irradiation the MED of each of the subjects was determined.
2. The 13 subjects were randomly divided in two groups of 10 and 3 subjects; tablets prepared according to example 1 (200 mg/tablet) were orally administered to the group of 10 subjects (12 tablets a day for 4 weeks). Ingestion time and method were at discretion of each subject (a daily dose of the “dry solid matter of preparation example 3” is 0.168 g). No preparations were given to the group of 3 subjects, in order to confirm that the MED did not vary during the test period. At the completion of the test, the MED was measured according to what described above.

Results

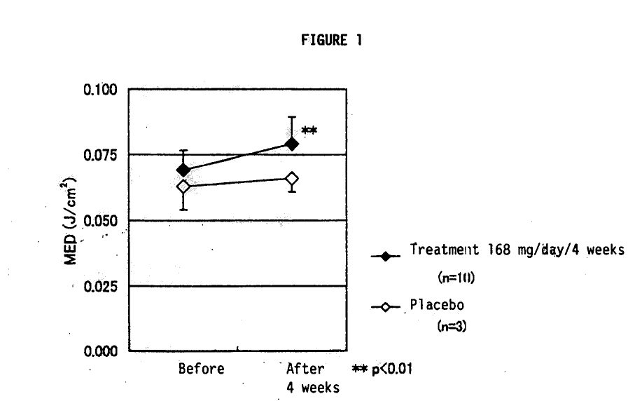

Test results are shown in FIG. 1. No difference in the MEDs before and after the test was observed in the reference group, while in the group that had been administered with the tablets prepared according to example 1 for four weeks, the MED significantly increased (p<0.01), i.e. resistance against UV rays increased and inflammation was prevented.

The following examples relate to other oral formulations containing the extract of the invention.

### Example 3

### Tablet

| | Amount |
|---|---|
| Ingredient | (weight %) |
| (1) “Dry solid matter of Preparation Example 3” | 50.0 |
| (2) Dextrin | 20.0 |
| (3) Crystalline cellulose | 23.0 |
| (4) Agar powder | 4.0 |
| (5) Aroma | 1.0 |
| (6) Sucrose fatty acid ester | 2.0 |

The ingredients were thoroughly mixed and formulated as tablets according to a standard procedure.

### Example 4

### Granular Formulation

| | Amount |
|---|---|
| Ingredient | (weight %) |
| (1) “Dry solid matter of Preparation Example 3” | 20.0 |
| (2) Starch | 30.0 |
| (3) Lactose | 49.0 |
| (4) Crystalline cellulose | 1.0 |

The ingredients were thoroughly mixed and formulated as a granular formulation according to a standard procedure.

### Example 5

### Soft Capsules

| | Amount |
|---|---|
| Ingredient | (weight %) |
| (1) “Concentrate of Preparation Example 1” | 30.0 |
| (2) Soybean oil | 25.0 |
| (3) Vitamin E | 20.0 |
| (4) Wheat germ oil | 15.0 |
| (5) Glycerin fatty acid ester | 5.0 |
| (6) Beeswax | 5.0 |

The ingredients were thoroughly mixed and formulated as soft capsules according to a standard procedure.

### Example 6

### Hard Capsules

| | Amount |
|---|---|
| Ingredient | (weight %) |
| (1) “Dry solid matter of Preparation Example 2” | 30.0 |
| (2) Powder sugar | 45.0 |
| (3) Dextrin | 24.0 |
| (4) Glycerin fatty acid ester | 1.0 |

The ingredients were thoroughly mixed and formulated as hard capsules according to a standard procedure.

### Example 7

### Drinkable Formulation

| | Amount |
|---|---|
| Ingredient | (weight %) |
| (1) “Dry solid matter of Preparation Example 3” | 1.5 |
| (2) Reduced maltose starch syrup | 20.0 |
| (3) Erythritol | 10.0 |
| (4) Citric acid | 1.0 |
| (5) Pure water | Balance |

The ingredients were mixed and formulated as a drinkable formulation according to a standard procedure.

### Example 8

### Jelly Formulation

| | Amount |
|---|---|
| Ingredient | (weight %) |
| (1) “Dry solid matter of Preparation Example 3” | 0.1 |
| (2) Dextrin | 24.0 |
| (3) Palatinose | 5.0 |
| (4) Gelatin | 1.0 |
| (5) Pectin | 0.5 |
| (6) Inositol | 5.0 |
| (7) Citric acid | 0.8 |
| (8) Ascorbic acid | 3.0 |
| (9) Nicotinic amide | 0.01 |
| (10) Pure water | Balance |

The ingredients were mixed and formulated as a jelly formulation according to a standard procedure.

### Example 9

### Chewing Gum

| | Amount |
|---|---|
| Ingredient | (weight %) |
| (1) “Dry solid matter of Preparation Example 2” | 5.0 |
| (2) Gum base | 25.0 |
| (3) Maltitol | 45.0 |
| (4) Mannitol | 20.0 |
| (5) Sorbitol | 5.0 |
| (6) Aroma | 1.0 |
| (7) Pure water | Balance |

The ingredients above were and formulated as a chewing gum according to a standard procedure.

### Example 10

### Soft Candy

| | Amount |
|---|---|
| Ingredient | (weight %) |
| (1) “Dry solid matter of Preparation Example 3” | 5.0 |
| (2) Granular sugar | 34.0 |
| (3) Starch syrup | 30.0 |
| (4) Gelatin | 10.0 |
| (5) Citric acid | 0.5 |
| (6) Tartaric acid | 0.3 |
| (7) Aroma | 1.0 |
| (8) Pure water | Balance |

The ingredients were thoroughly pulverized and mixed, and formulated as a gummy candy formulation according to a standard procedure.

1. A UV damage-preventing agent containing an aqueous part obtained by pressing olive fruits.

2. A UV damage-preventing agent containing an extract obtained by extracting an aqueous part and a solid part obtained by pressing olive fruits with water and/or an organic solvent.

3. A composition for the oral administration containing a UV damage preventing agent according to claim 1.

4. The composition according to claim 3, wherein the content of the extract obtained by extracting an aqueous part and a solid part obtained by pressing olive fruits with water and/or an organic solvent ranges from 0.01 to 70% by mass relative to the amount of the dry weight of the beverage composition.
